(19) United States
(12) Patent Application Publication
(10) Pub. No.: US 2010/0069679 A1
(43) Pub. Date: Mar. 18, 2010

(54) ACETYLENE TOLERANT HYDROFORMYLATION CATALYSTS

(75) Inventor: Thomas A. Puckette, Longview, TX (US)

Correspondence Address: BRETT L. NELSON;EASTMAN CHEMICAL COMPANY, 100 NORTH EASTMAN ROAD, KINGSPORT, TN, 37660-5075, US

(73) Assignee: Eastman Chemical Company, Kingsport, TN (US)

(21) Appl. No.: 12/209,626

(22) Filed: Sep. 12, 2008

(51) Int. Cl. B01J 31/02; C07C 45/50

(52) U.S. Cl. 568/454; 502/166

(57) ABSTRACT

Hydroformylation catalysts which can tolerate high levels of alkyne impurities in an olefin feed stream. The catalysts are composed of rhodium in combination with fluorophosphite ligands or rhodium in combination with certain bidentate ligands. Propionaldehyde has been produced by the hydroformylation of ethylene streams containing up to 1000 parts per million of acetylene without any loss of activity due to the presence of the acetylene. Ethylene contaminated with acetylene at 10,000 ppm can also be converted to propionaldehyde but some loss of activity occurs.

### BACKGROUND OF THE INVENTION

The hydroformylation of ethylene to prepare propionaldehyde is a well known and important industrial reaction. The preparation of propionaldehyde is accomplished by the reaction of ethylene with hydrogen and carbon monoxide in the presence of a rhodium containing catalyst under conditions of elevated temperature and pressure. Ethylene from commercial sources such as refineries and cracking plants frequently contains acetylene in varying amounts. Typically, the acetylene is removed from the ethylene stream by partial hydrogenation. However, during times of upset operation, the ethylene product may be contaminated with varying levels of acetylene which reduces the catalyst activity in a non-permanent manner.

The effects of acetylene impurities on the hydroformylation of olefins, such as ethylene, vary with the nature of the trivalent phosphorus ligand that is used to stabilize the rhodium catalyst. Strongly basic phosphine ligands are more susceptible to the reversible poisoning effects of acetylene. When the acetylene contamination is in the single digit part per million level, the negative effects of acetylene are small. However, when the acetylene levels exceed single digit numbers, the loss of activity can be catastrophic to the reaction. Therefore, it is highly desirable to have a catalyst system that can withstand the acetylene impurity spikes without loss of catalyst activity.

### BRIEF SUMMARY OF THE INVENTION

An embodiment according to the present invention concerns a process for the conversion of olefins into aldehydes. The process comprises contacting an olefin stream with a catalyst in a solvent to form at least one aldehyde, wherein the catalyst is a rhodium compound in combination with a ligand selected from the group consisting of a monodentate triorganophosphorus ligand, a bidentate organophosphorus ligand or a combination thereof, the ligand to rhodium molar ratio is from about 1:1 to about 1000:1, and said acetylene does not substantially reduce the conversion activity of the catalyst.

Another embodiment concerns a composition comprising a rhodium compound in combination with a ligand selected from the group consisting of a monodentate triorganophosphorus ligand, a bidentate organophosphorus ligand or a combination thereof.

### DETAILED DESCRIPTION

The present invention concerns certain catalyst compositions which can tolerate acetylene impurities in the olefin feed stream. By careful choice of the phosphorus ligand, a rhodium catalyst can be prepared that is unaffected by acetylene levels up to 1000 parts per million in the olefin feed and are still operational at acetylene levels of 2000, 5000, 7500 or even as; high as 10000 parts per million. These catalyst compositions are based on electron poor trivalent phosphorus ligands and some specific bidentate ligands.

The present invention presents a catalyst system that can make propionaldehyde from ethylene without sensitivity to acetylene. The value of this catalyst is that it can ride through upsets in the purity of the ethylene supply without having to take extraordinary measures to maintain reactor productivity.

The catalysts of the present invention allow for the preparation of aldehydes from olefin streams that intermittently contain alkyne impurities such as acetylene. The present invention also allows for the preparation of aldehydes from lower quality olefin streams without the requirement of an extra processing step to remove the alkyne impurities.

In the broadest description, this invention is a catalyst and a process for the conversion of ethylene that contains varying amounts of acetylene into propionaldehyde wherein the composition of the catalyst is a rhodium compound in combination with a monodentate triorganophosphorus ligand or a bidentate organophosphorus, ligand and a suitable solvent, and the process and the catalyst are unaffected by addition of acetylene to the feed stream at levels up to 1000 ppm.

As used herein, the phrase “substantially free” does not preclude a slight reduction in catalyst conversion activity, and in this regard, a reduction of less than about 10%, 5.0% or even 1.0% conversion activity over a period of at least about 3 hours, at least about 4 hours, or even at least about 5 hours is considered to be a process wherein conversion activity of the catalyst is not reduced.

Rhodium compounds that may be used as a source of rhodium for the active catalyst include rhodium(II) or rhodium(III) salts of carboxylic acids, examples of which include di-rhodium tetraacetate dihydrate, rhodium(II) acetate, rhodium(II) isobutyrate, rhodium(II) 2-ethylhexanoate, rhodium(II) benzoate and rhodium(II) octanoate. Also, rhodium carbonyl species such as Rh₄(CO)₁₂, Rh₆(CO)₁₆ and rhodium(I) acetylacetonate dicarbonyl may be suitable rhodium feeds. Additionally, rhodium organophosphine complexes such as tris(triphenylphosphine)rhodium carbonyl hydride may be used when the phosphine moieties of the complex fed are easily displaced by the ligands of the present invention. Less desirable rhodium sources are rhodium salts of strong mineral acids such as chlorides, bromides, nitrates, sulfates, phosphates and the like.

The concentration of the rhodium and ligand in the hydroformylation solvent or reaction mixture can vary. As mentioned hereinabove, a gram mole ligand:gram atom rhodium ratio of at least 1:1 normally is maintained in the reaction mixture. The absolute concentration of rhodium in the reaction mixture or solution may vary from 1 mg/liter up to 5000 mg/liter or more. When the process is operated within the practical conditions of this invention, the concentration of rhodium in the reaction solution normally is in the range of about 10 to 300 mg/liter.

We have found that the fluorophosphite ester compounds having the formula

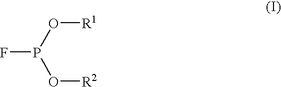

function as effective ligands when used in combination with transition metals to form catalyst systems for the processes described hereinabove. The hydrocarbyl groups represented by R¹ and R² may be the same or different, separate or combined, and are selected from unsubstituted and substituted alkyl, cycloalkyl and aryl groups containing a total of up to about 40 carbon atoms. The total carbon content of substituents R¹ and R² preferably is in the range of about 2 to 35 carbon atoms. Examples of the alkyl groups which R¹ and/or R² separately or individually can represent include ethyl, butyl, pentyl, hexyl, 2-ethylhexyl, octyl, decyl, dodecyl, octadecyl and various isomers thereof. The alkyl groups may be substituted, for example, with up to two substituents such as alkoxy, cycloalkoxy, formyl, alkanoyl, cycloalkyl, aryl, aryloxy, aroyl, carboxyl, carboxylate salts, alkoxycarbonyl, alkanoyloxy, cyano, sulfonic acid, sulfonate salts and the like. Cyclopentyl, cyclohexyl and cycloheptyl are examples of the cycloalkyl groups R¹ and/or R² individually can represent. The cycloalkyl groups may be substituted with alkyl or any of the substituents described with respect to the possible substituted alkyl groups. The alkyl and cycloalkyl groups which R¹ and/or R² individually can represent preferably are alkyl of up to about 8 carbon atoms, benzyl, cyclopentyl, cyclohexyl or cycloheptyl.

Examples of the aryl groups which R¹ and/or R² individually can represent include carbocyclic aryl such as phenyl, naphthyl, anthracenyl and substituted derivatives thereof. Examples of the carbocyclic aryl groups which R¹ and/or R² individually can represent the radicals having the formulas

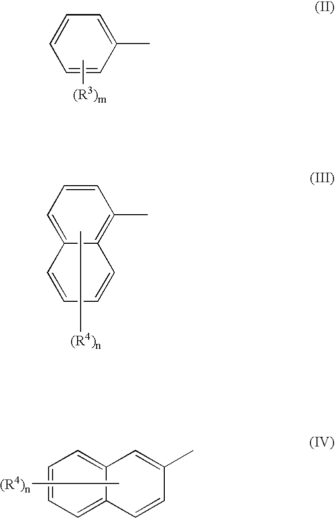

wherein R³ and R⁴ may represent one or more substituents independently selected from alkyl, alkoxy, halogen, cycloalkoxy, formyl, alkanoyl, cycloalkyl, aryl, aryloxy, aroyl, carboxyl, carboxylate salts, alkoxy-carbonyl, alkanoyloxy, cyano, sulfonic acid, sulfonate salts and the like. The alkyl moiety of the aforesaid alkyl, alkoxy, alkanoyl, alkoxycarbonyl and alkanoyloxy groups typically contains up to about 8 carbon atoms. Although it is possible for m to represent 0 to 5 and for n to represent 0 to 7, the value of each of m and n usually will not exceed 2. R³ and R⁴ preferably represent lower alkyl groups, i.e., straight-chain and branched-chain alkyl of up to about 4 carbon atoms, and m and n each represent 0, 1 or 2.

Alternatively, R¹ and R² in combination or collectively may represent a divalent hydrocarbylene group containing up to about 40 carbon atoms, preferably from about 12 to 36 carbon atoms. Examples of such divalent groups include alkylene of about 2 to 12 carbon atoms, cyclohexylene and arylene. Specific examples of the alkylene and cycloalkylene groups include ethylene, trimethylene, 1,3-butanediyl, 2,2-dimethyl-1,3-propanediyl, 1,1,2-triphenylethanediyl, 2,2,4-trimethyl-1,3-pentanediyl, 1,2-cyclohexylene, and the like. Examples of the arylene groups which R¹ and R² collectively may represent are given herein below as formulas (V), (VI) and (VII).

The divalent groups that R¹ and R² collectively may represent include radicals having the formula

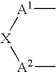

wherein each of A¹ and A² is an arylene radical, e.g., a divalent, carbocyclic aromatic group containing 6 to 10 ring carbon atoms, wherein each ester oxygen atom of fluorophosphite (I) is bonded to a ring carbon atom of A¹ and A²;
X is (i) a chemical bond directly between ring carbon atoms of A¹ and A²; or (ii) an oxygen atom, a group having the formula —(CH₂)_{y}— wherein y is 2 to 4 or a group having the formula

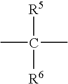

wherein R⁵ is hydrogen, alkyl or aryl, e.g., the aryl groups illustrated by formulas (II), (III) and (IV), and R⁶ is hydrogen or alkyl. The total carbon content of the group —C(R⁵)(R⁶)— normally will not exceed 20 and, preferably, is in the range of 1 to 8 carbon atoms. Normally, when R¹ and R² collectively represent a divalent hydrocarbylene group, the phosphite ester oxygen atoms, i.e. the oxygen atoms depicted in formula (I), are separated by a chain of atoms containing at least 3 carbon atoms.

Examples of the arylene groups represented by each of A¹ and A² include the divalent radicals having the formulas:

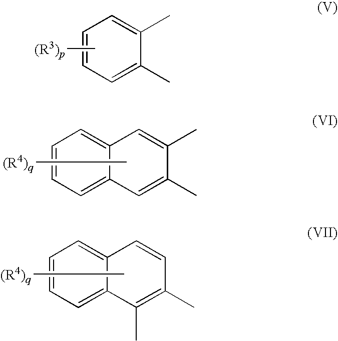

wherein R³ and R⁴ may represent one or more substituents independently selected from alkyl, alkoxy, halogen, cycloalkoxy, formyl, alkanoyl, cycloalkyl, aryl, aryloxy, aroyl, carboxyl, carboxylate salts, alkoxy-carbonyl, alkanoyloxy, cyano, sulfonic acid, sulfonate salts and the like. The alkyl moiety of such alkyl, alkoxy, alkanoyl, alkoxycarbonyl and alkanoyloxy groups typically contains up to about 8 carbon atoms. Although it is possible for p to represent 0 to 4 and for q to represent 0 to 6, the value of each of p and q usually will not exceed 2. R³ and R⁴ preferably represent lower alkyl groups, i.e., straight-chain and branched-chain alkyl of up to about 4 carbon atoms, and p and q each represent 0, 1 or 2.

Examples of fluorophosphite esters which are useful in the present invention include those which exhibit the best stability, are those wherein the fluorophosphite ester oxygen atoms are bonded directly to a ring carbon atom of a carbocyclic, aromatic group, e.g., an aryl or arylene group represented by any of formulas (II) through (VII). When R¹ and R² individually each represents an aryl radical, e.g., a phenyl group, in one embodiment, 1 or both of the ring carbon atoms that are in a position ortho to the ring carbon atoms bonded to the fluorophosphite ester oxygen atom are substituted with an alkyl group, especially a branched chain alkyl group such as isopropyl, tert-butyl, tert-octyl and the like. Similarly, when R¹ and R² collectively represent a radical having the formula

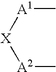

the ring carbon atoms of arylene radicals A¹ and A² that are in a position ortho to the ring carbon atoms bonded to the fluorophosphite ester oxygen atom are substituted with an alkyl group, preferably a branched chain alkyl group such as isopropyl, tert-butyl, tert-octyl and the like. In an embodiment, fluorophosphite esters have the general formula

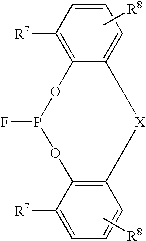

wherein each R⁷ is alkyl of 3 to 8 carbon atoms; each R⁸ is hydrogen, alkyl of 1 to 8 carbon atoms or alkoxy of 1 to 8 carbon atoms; and X is (i) a chemical bond directly between ring carbon atoms of each phenylene group to which X is bonded; or (ii) a group having the formula

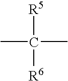

wherein each of R⁵ and R⁶ is hydrogen or alkyl of 1 to 8 carbon atoms.

The fluorophosphite esters of formula (I) may be prepared by published procedures or by techniques analogous thereto. See, for example, the procedures described by Riesel et al., J. Z. Anorg. Allg. Chem., 603, 145 (1991), Tullock et al., J. Org. Chem., 25, 2016 (1960), White et al., J. Am. Chem. Soc., 92, 7125 (1970) and Meyer et al., Z. Naturforsch, Bi. Chem. Sci., 48, 659 (1993) and in U.S. Pat. No. 4,912,155.

We have also found that certain bidentate ligands are also useful for the hydroformylation of ethylene containing varying amounts of acetylene. The ligands that have been found to be useful include BISBI and BIPHEPHOS.

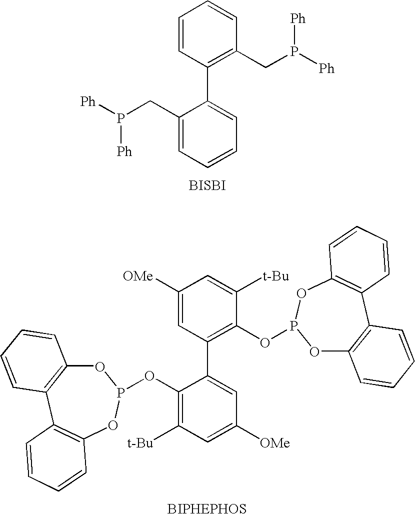

The ratio of gram moles phosphorus ligand to gram atoms transition metal can vary over a wide range, e.g., gram mole phosphorus:gram atom transition metal ratios of about 1:1 to 1000:1. For the rhodium-containing catalyst systems based on phosphite and fluorophosphite ligands, the gram mole phosphorus:gram atom rhodium ratio can be in the range of about 100:1 up to about 750:1, about 200:1 to about 400:1 or evenabout 250:1 to 300:1.

Different classes of phosphorus ligands such phosphines will exhibit very different behaviors and will require different phosphorus to rhodium molar ratios. Bidentate ligands which chelate strongly to the rhodium atom can be run at much lower ligand to rhodium molar ratios. For example, BISBI and BIPHEPHOS are bidentate phosphine and phosphite ligands respectfully and both have been shown to form stable, acetylene tolerant catalyst systems at ligand to rhodium molar ratios of about 1:1 to about 90:1 or from about 30:1 to about 80:1.

The hydroformylation reaction solvent may be selected from a wide variety of compounds, mixture of compounds, or materials that are liquid at the pressure at which the process is being operated. Such compounds and materials include various alkanes, cycloalkanes, alkenes, cycloalkenes, carbocyclic aromatic compounds, alcohols, esters, ketones, acetals, ethers and water. Specific examples of such solvents include alkane and cycloalkanes such as dodecane, decalin, octane, iso-octane mixtures, cyclohexane, cyclooctane, cyclododecane, methylcyclohexane; aromatic hydrocarbons such as benzene, toluene, xylene isomers, tetralin, cumene, alkyl-substituted aromatic compounds such as the isomers of diisopropylbenzene, triisopropylbenzene and tert-butylbenzene; crude hydrocarbon mixtures such as naphtha, mineral oils and kerosene; high-boiling esters such as 2,2,4-trimethyl-1,3-pentanediol diisobutyrate. The aldehyde product of the hydroformylation process also may be used. In one embodiment, a useful solvent is the higher boiling by-products that are naturally formed during the process of the hydroformylation reaction and the subsequent steps, e.g., distillations, that are required for aldehyde product isolation. The main criteria for the solvent are that it dissolves the catalyst and olefin substrate and that it does not act as a poison to the catalyst. In an embodiment, useful solvents for the production of volatile aldehydes, e.g., propionaldehyde and the butyraldehydes, are those that are sufficiently high boiling to remain, for the most part, in a gas sparged reactor. Solvents and solvent combinations that can be used in the production of less volatile and non-volatile aldehyde products include 1-methyl-2-pyrrolidinone, dimethyl-formamide, perfluorinated solvents such as perfluoro-kerosene, sulfolane, water, and high boiling hydrocarbon liquids as well as combinations of these solvents.

The reaction conditions used are not critical for the operation of the process and conventional hydroformylation conditions normally are used. The process requires that an olefin is contacted with hydrogen and carbon monoxide in the presence of the novel catalyst system described hereinabove. The process may be carried out at temperatures in the range of about 20° to 200° C., or reaction temperatures are from 50° to 135° C. or reaction temperatures ranging from 75° to 125° C.

The hydrogen:carbon monoxide molar ratio in the reactor likewise may vary considerably ranging from 10:1 to 1:10 and the sum of the absolute partial pressures of hydrogen and carbon monoxide may range from 0.3 to 36 bars absolute. The partial pressures of the hydrogen and carbon monoxide in the feed is selected according to the linear:branched isomer ratio desired. Generally, the partial pressure of hydrogen in the reactor can be maintained within the range of about 1.4 to about 13.8 bars absolute (about 20 to about 200 psia). The partial pressure of carbon monoxide in the reactor can be maintained within the range of about 1.4 to about 13.8 bars absolute (about 20 to about 200 psia) or from about 4 to about 9 bars absolute and may be varied independently of the hydrogen partial pressure. The molar ratio of hydrogen to carbon monoxide can be varied widely within these partial pressure ranges for the hydrogen and carbon monoxide. The ratios of the hydrogen to carbon monoxide and the partial pressure of each in the feed gas stream (often referred to as synthesis gas or syn gas) can be readily changed by the addition of either hydrogen or carbon monoxide to the synthesis gas stream.

The catalysts of the present invention can successfully hydroformylate acetylene contaminated ethylene to produce propionaldehyde without loss of activity. Acetylene levels of contamination up to 1000 ppm do not cause any loss of activity while levels of 10,000 ppm will cause some loss of activity.

Any of the known hydroformylation reactor designs or configurations may be used in carrying out the process provided by the present invention. Thus, a gas-sparged, vapor take-off reactor design as disclosed in the examples set forth herein may be used. In this mode of operation, the catalyst which is dissolved in a high boiling organic solvent under pressure does not leave the reaction zone with the aldehyde product taken overhead by the unreacted gases. The overhead gases then are chilled in a vapor/liquid separator to liquefy the aldehyde product and the gases can be recycled to the reactor. The liquid product is let down to atmospheric pressure for separation and purification by conventional technique. The process also may be practiced in a batchwise manner by contacting the olefin, hydrogen and carbon monoxide with the present catalyst in an autoclave.

A reactor design where catalyst and feedstock are pumped into a reactor and allowed to overflow with product aldehyde, i.e. liquid overflow reactor design, is also suitable. For example, high boiling aldehyde products such as nonyl aldehydes may be prepared in a continuous manner with the aldehyde product being removed from the reactor zone as a liquid in combination with the catalyst. The aldehyde product may be separated from the catalyst by conventional means such as by distillation or extraction and the catalyst then recycled back to the reactor. Water soluble aldehyde products, such as hydroxy butyraldehyde products obtained by the hydroformylation of allyl alcohol, can be separated from the catalyst by extraction techniques. A trickle-bed reactor design also is suitable for this process. It will be apparent to those skilled in the art that other reactor schemes may be used with this invention.

No special or unusual techniques are required for preparing the catalyst systems and solutions of the present invention, although, to obtain a catalyst of high activity, all manipulations of the rhodium and fluorophosphite ligand components be carried out under an inert atmosphere, e.g., nitrogen, argon and the like. The desired quantities of a suitable rhodium compound and ligand are charged to the reactor in a suitable solvent. The sequence in which the various catalyst components or reactants are charged to the reactor is not critical.

The hydroformylation reaction of ethylene to produce propionaldehyde is practiced on large industrial scale. The propionaldehyde serves as a feedstock for other useful chemicals including propionic acid and propanol.

The work described herein was conducted in a continuous operation bench unit as described in U.S. Pat. No. 5,840,647. The hydroformylation of ethylene to produce propionaldehyde was carried out in a vapor take-off reactor consisting of a vertically arranged stainless steel pipe having a 2.5 cm inside diameter and a length of 1.2 meters. The reactor was encased in an external jacket that was connected to a hot oil machine. The reactor had a filter element welded into the side down near the bottom of the reactor for the inlet of gaseous reactants. The reactor contained a thermowell arranged axially with the reactor in its center for accurate measurement of the temperature of the hydroformylation reaction mixture. The bottom of the reactor had a high pressure tubing connection that was connected to a cross. One of the connections to the cross permitted the addition of non-gaseous reactants such as octene-1 or make-up solvent, another led to the high-pressure connection of a differential pressure (D/P) cell that was used to measure catalyst level in the reactor and the bottom connection was used for draining the catalyst solution at the end of the run.

In the hydroformylation of ethylene in a vapor take-off mode of operation, the hydroformylation reaction mixture or solution containing the catalyst was sparged under pressure with the incoming reactants of ethylene, hydrogen, and carbon monoxide as well as any inert feed such as nitrogen. As propionaldehyde was formed in the catalyst solution, it and unreacted reactant gases were removed as a vapor from the top of the reactor by a side-port. The vapor removed was chilled in a high-pressure separator where the propionaldehyde product was condensed. The uncondensed gases were let down to atmospheric pressure via the pressure control valve. These gases passed through a series of dry-ice traps where any other aldehyde product was collected. The product from the high-pressure separator was combined with that of the traps, and was subsequently weighed and analyzed by standard gas/liquid phase chromatography (GLC) techniques for the net weight and propionaldehyde product.

The gaseous feeds to the reactor were fed to the reactor via twin cylinder manifolds and high-pressure regulators. The hydrogen passed through a mass flow controller and then through a commercially available DEOXO® (available from Engelhard Inc.) catalyst bed to remove any oxygen contamination. The carbon monoxide passed through an iron carbonyl removal bed (as disclosed in U.S. Pat. No. 4,608,239), a similar DEOXO® bed heated to 125° C., and then a mass flow controller. Nitrogen could be added to the feed mixture as an inert gas. Nitrogen, when added, was metered in and then mixed with the hydrogen feed prior to the hydrogen DEOXO® bed. Ethylene was fed to the reactor from cylinders and was controlled using a mass flow meter. All gases feeds were passed through a preheater prior to entering the reactor.

This invention can be further illustrated by the following examples of preferred embodiments thereof, although it will be understood that these examples are included merely for purposes of illustration and are not intended to limit the scope of the invention unless otherwise specifically indicated.

### EXAMPLES

A bench unit catalyst was prepared from rhodium 2-ethylhexanoate, dioctylphthalate solvent, and the phosphorus ligand as noted in the table. The catalyst was charged to the continuous bench unit reactor, heated to temperature and fed the reactants. The unit was operated for three hours using a pure ethylene feed as the olefin feed. The catalyst activity in the third hour was taken as a base point for further comparisons and then the olefin feed was switched from pure ethylene to ethylene containing 1000 ppm acetylene. The catalyst activity for the ensuing hours was observed and recorded in Table I.

The data in Table I shows that phosphine based catalysts are strongly negatively affected by acetylene when compared with the fluorophosphite based catalysts of the present invention. In particular, the more basic ligands such as tricyclohexylphosphine and tribenzylphosphine are more strongly affected. The abbreviations used in the table are: TBZP is tribenzylphosphine; TPP is triphenylphosphine; TCHP is tricyclohexylphosphine; and Ethanox 398™ is 2,2′-Ethylidene-bis-(4,6-di-tert-butylphenyl)fluorophosphite.

Lines 3 and 4 in Table I illustrate the value of and the stabilizing effect of increasing the ligand to rhodium molar ratio.

TABLE I
Hydroformylation of Ethylene Containing 1000 Parts Per Million Acetylene

| | | | | Catalyst Activity, grams of | | | |
|---|---|---|---|---|---|---|---|
| | Millimole | | | propionaldehyde | | | |
| | of ligand | Rhodium | Ligand/Rh | (% of hour 3 activity retained) | | | |
| Ligand | used | milligrams | mole ratio | Hour 3 | Hour 4 | Hour 5 | Reference |
| Ethanox 398 ™ | 8.0 | 3.0 | 275 | 83.1 | 84.3 (101%) | 84.1 (101%) | X-29055- |
| | | | | | | | 022 |
| Ethanox 398 ™ | 8.0 | 3.0 | 275 | 60.6 | 57.1 (94%) | 55.2 (91%) | X-29055- |
| | | | | | | | 052 |
| 2,2′-methylidene- | 8.0 | 3.0 | 274 | 67.0 | 70 (104%) | 67.4 (101%) | X-29055- |
| bis- | | | | | | | 092 |
| (6-di-tert-butyl-4- | | | | | | | |
| dimethylphenyl) | | | | | | | |
| fluorophosphite | | | | | | | |
| 2,2′-methylidene- | 1.2 | 3.0 | 41 | 126.3 | 106.1 | 31.4 | X-29055- |
| bis- | | | | | | | 090 |
| (6-di-tert-butyl-4- | | | | | | | |
| dimethylphenyl) | | | | | | | |
| fluorophosphite | | | | | | | |
| TBzP, | 5.3 | 10.0 | 55 | 44.6 | 36.5 (82%) | 19.4 (43%) | X-29055- |
| | | | | | | | 024 |
| TCHP | 5.3 | 10.0 | 55 | 25.0 | 7.6 (30%) | 2.4 (10%) | X-29055- |
| | | | | | | | 028 |
| TPP | 17.0 | 5.0 | 350 | 96.0 | 32.8 (34%) | 7.2 (8%) | X-29055- |
| | | | | | | | 108 |
| TPP | 29.1 | 5.0 | 599 | 162.5 | 48.5 (30%) | 19.4 (12%) | X-29055- |
| | | | | | | | 114 |
| TPP | 40.0 | 6.0 | 687 | 100.8 | 90.3 (90%) | 60.2 (60%) | X-29055- |
| | | | | | | | 026 |
| TCHP & TBP | 2.7 | 20 | 28 | 20.7 | 13.4 (65%) | 6.2 (30%) | X-29055- |
| | mmole | | | | | | 038 |
| | each | | | | | | |

All runs were made at 105° C., 260 psig with 1:1 syn gas (192 psia), N₂ (29 psia), C₂H₄ (54 psia) with a total gas flow of 9.6 liters/minute in 190 milliliters of DOP solvent

The table shows that all of the ligands other than the fluorophosphites (Ethanox 398™ and 2,2′-methylidene-bis-(6-di-tert-butyl-4-dimethylphenyl)fluorophosphite) show some degree of catalyst activity loss with the introduction of acetylene. The severity of the catalyst activity loss is a function of the ligand. The strongly basic ligand, TCHP, shows the most significant loss of activity. The triphenylphosphine based catalyst systems shows less of a loss of activity at higher ligand loadings as taught by Fell and Beutler and also in U.S. Pat. No. 5,675,041.

We have also found that certain bidentate ligands can be used for the hydroformylation of ethylene and that acetylene at 1000 ppm has no effect on these bidentate ligands. The entries in table II are two bidentate ligands. BISBI is the bidentate phosphine ligand 1,1′-(2,2′-bisdiphneylphosphinomethyl)biphenyl. The BIPHEPHOS ligand is a bidentate bis phosphite ligand, 2,2′-Bis[(1,1′-biphenyl-2,2′-diyl)phosphite]-3,3′-di-tert-butyl-5,5′-dimethoxy-1,1′-biphenyl; CAS registry number 121627-17-6. Neither of these bidentate ligands was strongly affected by the acetylene contaminant at 1000 ppm. The experimental work of Table I above was repeated using an ethylene feed that was spiked to 1,000 ppm of acetylene. The reactions were monitored for two half hour intervals after the introduction of the acetylene containing feed. The data from these runs is presented in Table II.

TABLE II
Acetylene Tolerance of Bidentate Ligand based Catalysts

| | | | Catalyst Activity, grams of | | | |
|---|---|---|---|---|---|---|
| | | | propionaldehyde | | | |
| | Millimole of | Rhodium | (% of hour 3 activity retained) | | | |
| Ligand | ligand used | milligrams | Hour 3 | Hour 3.5 | Hour 4.0 | Reference |
| BISBI | 3.0 | 4.0 | 75.3 | 71.5 (95%) | 75.3 (100%) | X-29055- |
| | | | | | | 036 |
| BIPHEPHOS | 2.5 | 3.0 | 48.4 | 52.2 (107%) | 51.2 (105%) | X-29055- |
| | | | | | | 040 |

All runs were made at 105° C., 260 psig with 1:1 syn gas (192 psia), N₂ (29 psia), C₂H₄ (54 psia) with a total gas flow of 9.6 liters/minute in 190 milliliters of DOP solvent

The experimental work of Table I above was repeated using an ethylene feed that was spiked to 10,000 ppm of acetylene. The reactions were monitored for two half hour intervals after the introduction of the acetylene containing feed. The acetylene at 10,000 ppm was sufficient to have a negative effect on all catalysts including the Ethanox 398™ based catalyst. However, the Ethanox 398™ based catalyst shows the greatest retention of activity after two hours of operation with the 10,000 ppm acetylene feed. The results of these experiments are presented in the table below.

TABLE II
Hydroformylation of Ethylene Containing 10,000 Parts Per Million of Acetylene

| | | | | Catalyst Activity, grams | | | |
|---|---|---|---|---|---|---|---|
| | | Ligand | | of propionaldehyde | | | |
| Ligand, | Millimole | to Rh | | (% of hour 3 activity | | | |
| Reaction | of ligand | Molar | Rhodium | retained) | | | |
| Temperature | used | Ratio | milligrams | Hour 3 | Hour 3½ | Hour 4 | Reference |
| TPP, 105 C. | 17.0 | 87 | 5 | 94.8 | 32.9 (69%) | 7.0 (15%) | X-29055- |
| | | | | | | | 108 |
| TPP, 105 C. | 29.1 | 150 | 5 | 78.7 | 28.8 (73%) | 9.1 (23%) | X-29055- |
| | | | | | | | 112 |
| TPP, 115 C. | 29.1 | 150 | 5 | 162.5 | 48.5 (60%) | 19.4 (24%) | X-29055- |
| | | | | | | | 114 |
| Ethanox | 8.0 | 412 | 2 | 81.5 | 22.0 (54%) | 15.7 (39%) | X-29055- |
| 398 ™, 120 C. | | | | | | | 116 |
| Ethanox | 8.0 | 274 | 3 | 85.4 | 79.0 (93%) | 38.2 (45%) | X-29055- |
| 398 ™, 110 C. | | | | | | | 098 |

All runs were made at 260 psig with 1:1 syn gas (192 psia), N₂ (29 psia), C₂H₄ (54 psia) with a total gas flow of 9.6 liters/minute in 190 milliliters of DOP solvent

The invention has been described in detail with particular reference to preferred embodiments thereof, but it will be understood that variations and modifications can be effected within the spirit and scope of the invention.

I claim:

1. A process for the conversion of olefins into aldehydes, comprising
contacting an olefin stream with a catalyst in a solvent to form at least one aldehyde,
wherein
the catalyst is a rhodium compound in combination with a ligand represented by the following formula: 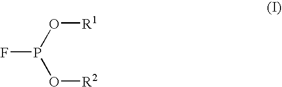
wherein R¹ and R² are the same or different, contain a combined total of up to about 40 carbon atoms, and are selected from the group consisting of an alkyl group, a cycloalkyl group, and an aryl group,
the olefin stream contains up to about 1,000 ppm acetylene,
the ligand to rhodium molar ratio is from about 1:1 to about 1000:1, and
said acetylene does not substantially reduce the conversion activity of the catalyst.

2. (canceled)

3. (canceled)

4. The process according to claim 1, wherein the ligand to rhodium molar ratio is from about 10:1 to about 750:1.

5. The process according to claim 4, wherein the ligand to rhodium molar ratio is from about 50:1 to about 400:1.

6. (canceled)

7. (canceled)

8. The process according to claim 1, wherein the conversion activity of the catalyst is reduced less than 10%.

9. The process according to claim 8, wherein the conversion activity of the catalyst is reduced less than 5.0%.

10. The process according to claim 9, wherein the conversion activity of the catalyst is reduced less than 1.0%.

11. The process according to claim 1, wherein the olefin is ethylene.

12. The process according to claim 1, wherein the aldehyde is propionaldehyde.

13. (canceled)

14. The process according to claim 1, wherein the alkyl group is selected from the group consisting of ethyl, butyl, pentyl, hexyl, 2-ethylhexyl, octyl, decyl, dodecyl, octadecyl and various isomers thereof.

15. The process according to claim 14, wherein the alkyl groups are substituted with up to two substituents selected from the group consisting of alkoxy, cycloalkoxy, formyl, alkanoyl, cycloalkyl, aryl, aryloxy, aroyl, carboxyl, carboxylate salts, alkoxycarbonyl, alkanoyloxy, cyano, sulfonic acid, and sulfonate salt.

16. The process according to claim 1, wherein the cycloalkyl group is selected from the group consisting of benzyl, cyclopentyl, cyclohexyl and cycloheptyl.

17. The process according to claim 1, wherein the aryl group is selected from the group consisting of phenyl, naphthyl, anthracenyl and substituted derivatives thereof.

18. A process for the conversion of olefins into aldehydes, comprising
contacting an olefin stream with a catalyst in a solvent to form at least one aldehyde,
wherein
the catalyst is a rhodium compound in combination with a ligand represented by one of the following formulas: 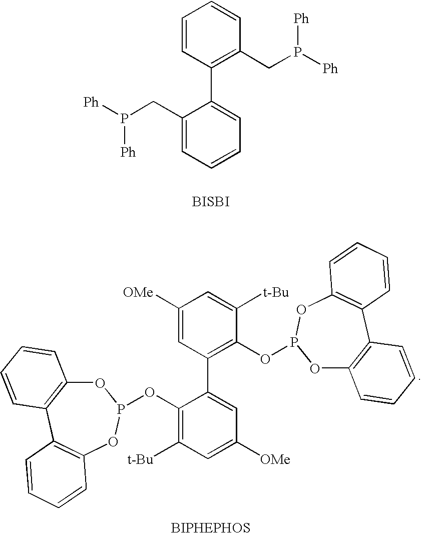
the olefin stream contains up to about 1,000 ppm acetylene.
the ligand to rhodium molar ratio is from about 1:1 to about 1000:1, and
said acetylene does not substantially reduce the conversion activity of the catalyst.

19-25. (canceled)

26. The process according to claim 18, wherein the ligand to rhodium molar ratio is from about 1:1 to about 90:1.

27. The process according to claim 26, wherein the ligand to rhodium molar ratio is from about 3:1 to about 30:1.

28. The process according to claim 18, wherein the olefin is ethylene.

29. The process according to claim 18, wherein the aldehyde is propionaldehyde.
